(19) United States
(12) Patent Application Publication
(10) Pub. No.: US 2011/0319558 A1
(43) Pub. Date: Dec. 29, 2011

(54) METHOD FOR COATING A FLOOR

(76) Inventors: Nicola Rehnberg, Perstorp (SE); Curt Persson, Malmo (SE); Ronny Lindell, Klagerup (SE); Nils Erik Persson, Sodra Sandby (SE)

(21) Appl. No.: 13/129,138

(22) PCT Filed: Oct. 16, 2009
(86) PCT No.: PCT/SE2009/051179

§ 371 Date: Jun. 20, 2011

(30) Foreign Application Priority Data
Nov. 17, 2008 (SE) 0850078-7

(51) Int. Cl. C09D 175/04; C07F 7/02; C07C 265/14; C08F 2/48; C08J 7/04

(52) U.S. Cl. 524/590; 427/508; 427/515; 560/359; 560/355; 556/465; 556/489

(57) ABSTRACT

A method for coating a floor, wherein said method comprises applying an aqueous dispersion comprising polyurethane, an acrylate or a hybride or mixture thereof; drying said coating and subjecting the physically dry coating to ultraviolet radiation whereby a cured coating is obtained. Also claimed is reactive additives suitable for use in the methods as well as the floor itself.

### FIELD OF THE INVENTION

The present invention relates to methods for coating a floor, a reactive additive for use in the methods as well as the floor itself.

### BACKGROUND OF THE INVENTION

Traditionally, floors have been protected by coatings.

Curing of a coating, that is formation of chemical bonds after a coating such as a paint, a varnish, or a lacquer has been applied to a surface, results in greatly improved properties. The oldest forms of curing coatings are derived from vegetable oils. Highly unsaturated oils, such as flaxseed oil, may be used as such but natural oils, such as sunflower oils and safflower oils, are frequently siccativated and further modified before being used as coatings. Blown oils or otherwise molecular weight increased products are more common. Alkyds are a special form of oil derived coatings. They have improved performance in comparison with oils or modified oils. They are however of high viscosity and are commonly diluted with suitable solvents.

The curing of oils and alkyds takes long time. To achieve a cured coating with maximum properties usually takes as long time as several days or even weeks depending on the coating.

Solvent free and rapidly curing coatings exist and may be based on acrylates (100% solids acrylate). The rapid curing is initiated by electromagnetic radiation and completed within fractions of a second. Examples of such coatings are rapid printing inks which are known since several decades. A typical coating formulation is composed of a high molecular weight oligomer, a multifunctional acrylate, a diluent such as a difunctional acrylate, an initiator and optionally a synergist. This type of coating is used industrially for the protection of e.g. wooden objects for furniture. A starting formulation for wood coating may be found in “Chemistry @ Technology of UV EB formulation for coatings, Inks Paints, Vol 2, p133, Ed P. K. T. Oldring, 1991, London, ISBN 0.947798 10.2. This type of coating has some limited utility in the protection of installed mineral flooring such as concrete, terrazzo, wood or plastics. Example of a technique presently used is disclosed in WO2004073894. WO2004073894 discloses a traditional, not physically drying, 100% solids acrylate coating applied to the floor and cured with a mobile UV-lamp. A wider use is prohibited for example due to complicated handling. Additionally, the toxic nature of low molecular weight components reduce its usability. For example, the wet surface may not be used for traffic and some part of the coating may not cure, and may remain wet for a very long time, and if low molecular weight components are used, such components will be absorbed by the flooring matrix. Volatile low molecular weight components deposited on the flooring matrix may cause a health problem due to their potential toxicity. Consequently, there are several drawbacks with the method disclosed in WO2004073894.

An alternative to 100% solids acrylate coatings, described above, are radiation curing aqueous dispersions, e.g. based on polyurethanes. They are used industrially for the coating of various objects, such as furniture and kitchen cabinets.

The traditional systems and techniques used in coating floors all suffer from different drawbacks such as a long drying time, stickiness and giving a floor coating of low quality. Consequently there is a need for a method or technique that improves the traditional systems and techniques.

### SUMMARY OF THE PRESENT INVENTION

An object of the present invention relates to a technique or method that is designed to meet the demands on protection of floors and in particular to a technique or method that improves the traditionally used methods for protection of a floor, such as coating of a floor. An object of the present invention relates to a method for coating a floor where the time to obtain a cured floor coating is reduced compared to methods traditionally used within the floor industry and where the properties of the coating is as good as or better than traditional coatings. Additionally, an object of the present invention relates to a method for coating a floor where the following are achieved by the method of the present invention: the time to obtain a cured floor is reduced compared to traditional coating methods used within the floor industry, and the method may result in a floor coating of high quality, and said method is providing a safe working environment with minimal risk of sensitisation and/or exposure to volatile organic components. These and other objects are achieved by the method according to the present invention.

Objects of the present invention are achieved by a method for coating a floor, wherein said method comprises applying a coating based on one or more radiation curable aqueous dispersion(s) comprising one or more polymerisable functional group(s) to a floor; drying said coating to obtain a physically dry coating; and subjecting the physically dry coating to electromagnetic radiation, whereby a cured coating is obtained.

In one aspect of the present invention the radiation curable aqueous dispersion having one or more polymerisable functional group(s) is a radiation curable polyurethane dispersion or a radiation curable acrylate dispersion or a hybride or a mixture thereof.

In one aspect of the present invention, one or more polymerisable functional group(s) is selected from acrylates, methacrylates, methacrylamides, and other vinyl derivatives, and thiolene components.

In one aspect of the present invention, the floor is cleaned and/or undressed before applying said coating.

In one aspect of the present invention, an additive is added to said coating, said additive may be a reactive additive, such as an isocyanate based reactive additive or an aziridine based reactive additive.

In one aspect of the present invention, the reactive additive has both isocyanate and alkoxysilane groups, or isocyanate and vinyl groups.

In one aspect of the present invention the additive is an additive promoting adhesion.

In one aspect of the present invention the reactive additive is a silicon derivative, such as a polymerisable silicon derivative, for example a radically polymerisable silicon derivative such as vinyltrimethoxysilane, 3-(methacryloyloxy)propyltrimethoxysilane, or 3-(methacryloyloxypropyl)-triethoxysilane; or a silicon product having reactive funtionalities, such as silica sol treated with an unsaturated alkoxysilane or a mercaptosilane such as vinyltrimethoxysilane, 3-(methacryloyloxy)propyltrimethoxysilane, 3-(methacryloyloxypropyl)triethoxysilane, or 3-(mercaptopropyl)-trimethoxysilane.

In one aspect the invention relates to a reactive additive. The reactive additive is used in the methods according to the invention and is a poly- or oligoisocyanate based reactive additive having one or more additional reactive group(s).

In one aspect the floor is selected from biologically derived flooring materials, such as woods, cork, bamboos or mineral derived flooring materials, such as concrete, terrazzo, ceramic tile, grout, plastics such as vinyl plastics.

Objects of the present invention are achieved by a coated floor obtainable by the above method for coating a floor.

Objects of the present invention are achieved by a coated floor obtainable by providing a floor; providing a coating based on a radiation curable aqueous dispersion comprising one or more polymerisable functional group(s); applying said coating to a floor, drying said coating to obtain a physically dry coating; and subjecting the physically dry coating to electromagnetic radiation.

### SHORT DESCRIPTION OF THE DRAWINGS

FIG. 1 illustrates the average time to obtain a cured coating obtained according to a traditional procedure using a two component lacquer made from a polyurethane dispersion with an associated isocyanate hardener (2k PUD) as reactive additive.

FIG. 2 illustrates a typical time to obtain a cured coating obtained according to a method of the present invention.

### DETAILED DESCRIPTION OF THE PRESENT INVENTION

The present invention relates to a method for coating a floor where the time to obtain a cured coating is reduced compared to methods traditionally used within the floor industry, said method results in the properties of the coating being as good as or better than traditional coatings.

According to the present invention a coating based on a radiation curable aqueous dispersion, such as a polyurethane or acrylate dispersion or hydbrid or a mixture thereof, is used to protect a floor. The radiation curable aqueous dispersion is formulated to a coating, i.e. a finish, a lacquer, a polish or a varnish, using different additives. Non-limiting examples of additives that may be used are film formation agents, preservatives, initiators, stabilisators, rheological modifier, surface modifying surfactants, flatting agents, waxes, defoamers, and wetting agents. A coating according to the invention comprising a radiation curable aqueous dispersion possesses several valuable properties such as physical drying, adequate mechanical properties before cure, good mechanical properties and good chemical resistance after cure, easy handling and a very rapid cure which may be beneficial for coating of a substrate, such as a floor. According to the present invention the above mentioned coating may contain one or more suitable additives. The additive may be selected from poly- or oligoisocyanate based reactive additives, aziridine based reactive additives, silicon reactive additives and carbodiimide reactive additives or any combination thereof. Said additive provides an alternative mood of cross-linking promoting important properties such as chemical and mechanical resistance and adhesion. In one aspect of the present invention, the additive is added to promote adhesion of the coating to the substrate. This is in particluar relevant for substrate known to be difficult to coat such as an industrially UV cured surface. The reactive additive may also advantageously be used for conventional substrates. For areas not cured by electromagnetic radiation, or that by mistake has not been cured, the invention offers a an advantage in that chemical and mechanical properties become adequate. The reactive additive in the form of a reactive additive, is according to the invention, a slower curing system than the radiation curing system but over time, such as 1 to 3 days (or longer), depending on factors within the capability of a skilled person to determine, such as climate factors, the properties in the areas that did not cure by electromagnetic radiation obtained adequate properties. Such properites are pendulum hardness, scratch resistance, black heel mark resistance, wear resistance and chemical resistance (water and other chemical resistance).

The silicon reactive additive may be selected from the group of silicon derivatives, such as a polymerisable silicon derivative, for example a radically polymerisable silicon derivative; or silicon products with reactive functionalities, such as silica sol treated with an unsaturated alkoxysilane or a mercaptosilane. In one embodiment the radically polymerisable silicon derivative is selected from 3-(trimethoxysilyl)propyl methacrylate; 3-(ethoxysilyl)propyl methacrylate; or an adduct of an aminosilane and an acrylate. Said aminosilane may be selected from the group consisting of 3-(trimethoxysilyl)propylamine, 3-(triethoxysilyl)propylamine and (dimethoxymethylsilyl)methylamine. Said acrylate may be selected from the group consisting of tripropylene glycol diacrylate, trimethylolpropane triacrylate and pentaerythritol pentaethoxylate tetraacrylate. In one embodiment the silica sol, treated with an unsaturated alkoxysilane or a mercaptosilane, is selected from Bindzil 30/220, Bindzil 305/220, Bindzil 30/360, Bindzil 40/130, Nyacol 1430 LS, or Nyacol DP 5110. The unsaturated alkoxysilane may be selected from vinyltrimethoxysilane, vinyltriethoxysilane, allyltrimethoxysilane, 3-(methacryloyloxy)propyltrimethoxysilane, 3-(methacryloyoloxy)propyltriethoxysilane, (methacyloxymethyl)-methyldimethoxysilane. The mercaptosilane may be selected from 3-mercaptopropyltrimethoxysilane.

In one embodiment the reactive additive is selected from poly- or oligoisocyanates, or polymers of a diisocyanate such as 1,6-diisocyanatohexane, toluene diisocyanate, isophorone diisocyanate or derivaitves thereof such as those exemplified in U.S. Pat. No. 4,663,377, U.S. Pat. No. 5,200,489, U.S. Pat. No. 5,252,696 or available under trade names such as Tolonate HDB, Tolonate HDT, and Tolonate IDT, Desmodur N3300, Desmodur N3600, Bayhydur 3100, Bayhydur VPLS2319, Bayhydur XP2487/1, Bayhydur XP2570, Bayhydur VP LS 2150, Easaqua X M 502, Bona Naturale Hardener

In one embodiment the poly- or oligoisocyanate based additive comprises one or more additional reactive group(s).

In one embodiment the one or more additional reactive group(s) is selected from an acrylate group and/or a silyl group.

The aziridine reactive additive may be selected from multi functional aziridines such as Bona Crosslinker.

The isocyanate based reactive additive or aziridine based reactive additive, as described above, has in some embodiments of the present invention been modified with a vinyl and/or silyl derivative. Such modification result in the reactive additive becoming UV-polymerizable or able to interact with silicon derivatives such as sols, silicates, particles or metal or metal oxides such as titanium dioxide or materials such as titanium dioxide, titanates, or aluminates or zirconium derivatives. In one embodiment, a modified isocyanate based reactive additive is an addition product of a poly- or oligoisocyanate derivative and a vinyl compound with an isocyanate reactive group. The poly- or oligoisocyanate may be selected from an oligomer or polymer of 1,6-diisocyanatohexane, toluene diisocyanate, isophorone diisocyanate, Tolonate HDB, Tolonate HDT, and Tolonate IDT, Desmodur N3300, Desmodur N3600Bayhydur 3100, Bayhydur VPLS2319, Bayhydur XP2487/1, Bayhydur XP2570, Bayhydur VP LS 2150, Bona Naturale Hardener; and the vinyl compound with an isocyanate reactive group may be selected from 2-hydroxyethyl acrylate or methacrylate, 2-hydroxypropyl acrylate or methacrylate, 4-hydroxybutyl acrylate or methacrylate, or glycerol diacrylate or dimethacrylate, or pentaerythritol triacrylate or trimethacrylate or a polyalkylene oxide derivative of acrylic or methacrylic acid such as triethylene glycol or tripropylene glycol monoacrylate or monomethacrylate, or hexaethylen glycol or hexapropylene glycol acrylate or methacrylate, or decaethylene glycol or decapropylene glycol acrylate or methacrylate, or mixtures such as Bisomer PEM63P, or a caprolactone derivative such as Bisomer Pemcure 12A. The above mentioned silyl derivative may be selected from 3-(trimethoxysilyl)propylamine, 3-(triethoxysilyl)propylamine, an adduct of an aminosilane derivative and an unsaturated compound. Said aminosilane may be selected from 3-(trimethoxysilyl)propylamine, or 3-(triethoxysilyl)propylamine and said unsaturated compound may be selected from butyl acrylate, dimethyl maleate, dibutyl maleate, or bis(2-ethylhexyl) maleate. In one embodiment the reactive additive is a trimer of diisocyanate.

The additive may be added at any suitable time but it is preferred that it is added before applying the coating to a substrate, such as a floor.

The present invention also relates to a method for protecting a floor, such as coating a floor. Examples of floors are biologically derived flooring substrates such as wooden flooring, cork flooring, linoleum flooring, bamboo flooring, and mineral derived flooring such as concrete, terrazzo, ceramic tile, grout, plastics such as vinyl plastics. In one embodiment the floor is selected from wooden flooring, cork flooring and bamboo flooring.

In one embodiment of the invention the method is time saving compared to conventional methods.

In one embodiment of the invention the method results in a cured coating of high quality.

The method of the present invention is beneficially compared to conventional methods as one or more of the above mentioned benefits are provided, i.e. being time saving and resulting in a cured coating of high quality.

The method according to the present invention is beneficial in terms of providing a safe working and use environment with minimal risk of sensitisation and/or exposure to volatile organic components, for example low molecular weight components that may be low molecular weight acrylates and epoxides etc.

In one aspect, the present invention relates to a method for the protection such as coating of flooring; such as biologically derived flooring such as wooden flooring, cork flooring, linoleum flooring, bamboo flooring, and mineral derived flooring such as concrete, terrazzo, ceramic tile, grout, plastics such as vinyl plastics, wherein the floor is coated with a coating based on a radiation curable polyurethane or acrylate dispersion able to develop the desired properties, such as scratch resistance, black heel mark resistance, wear resistance and chemical resistance. Some of these properties are present after physical drying but are not fully developed until the coating is UV-cured (Ultraviolet) by an electromagnetic radiation source.

In one embodiment of the present invention a method for the protection of a floor may be initiated by preparation of the floor. Preparation of the floor may include any type of cleaning such as vacuum cleaning, wet cleaning using water and a detergent or any other mean of making the floor free of dust and dirt. Additionally, preparation of the floor may include sanding of the floor, for example if the floor is heavily worn. The preparation may also involve any other way of undressing the floor. The preparation step may or may not be carried out depending on the judgement of the skilled person.

In one embodiment of the invention, a coating, such as a wet coating, is applied to a floor using conventional techniques and tools such as roller, squeegee, pad, brush or spray or any other suitable technique known to a person skilled in the art. After application of the coating, it is left to dry and when the water has evaporated to a certain degree, such that the coating is sufficiently dry to allow walking or moving for example a radiation device on the coating without damaging said coating, a physically dry coating remains. Properties of the physically dried coating may be determined by analyzing properties such as pendulum hardness, scratch resistance, black heel mark resistance, wear resistance and chemical resistance of the coating. A person skilled in the art may also determine that the physically dry coating is obtained by visual inspection and/or by touching the coating. Any of these parameters may be used individually or in any combination to determine if or when the desired properties of a physically dry coating has been obtained.

In one embodiment of the invention one or several layers of coating may be applied, said coating may be a protective coating. Each layer may or may not be radiation curable. In one embodiment of the invention a top coat in one or several layers may be applied, which may be radiation curable. According to the present invention one or more layers of coating may be applied to the floor, and at least one of the coating layers must be a coating based on a radiation curable aqueous dispersion, such as a polyurethane or acrylate dispersion or a mixture or hybride thereof.

In some embodiments, a primer is applied as one layer. Non-limiting examples of suitable primers are selected from Bona Prime Classic, Bona Prime Intense, UV curable oils, such as Dry max grundierung and Dry max-öl. Alternatively, primers based on a radiation curable aqueous dispersion, such as polyurethane or acrylate dispersions, may be used.

A physically dry coating means a non-sticky and/or thumb dry coating which is possible to use, such as performing certain actions, such as touching and using it without damaging the coating, and in particular not damaging the surface of the coating, without the final properties of the cured coating being developed. Said actions may for example involve moving, such as rolling, an electromagnetic radiation apparatus on the coating of the floor or walking on the coating of the floor without damaging its appearance and/or properties.

A physically dry coating is defined as above but may also be determined by analyzing properties of the coating such as pendulum hardness, scratch resistance, black heel mark resistance, wear resistance and chemical resistance. Any of these parameters may be used individually or in any combination to determine if the desired properties of a physically dry coating has been obtained. A person skilled in the art is able to determine and analyze when a coating is physically dry without the use of any particular equipment. The cured coating is obtained by electromagnetic radiation.

A cured coating is a coating to which electromagnetic radiation have been applied, allowing cross-linking, i.e., covalent bond formation between individual molecules of the physically dry coating to take place. Radiation is applied during a suitable time, at a suitable distance and by using an electromagnetic radiation device, such as a UV lamp having a suitable effect. The parameters necessary to obtain a cured coating are all known and possible to determine for a person skilled in the art. The cured coating is obtained within fractions of a second.

By controlling the effect output from the electromagnetic radiation source, the distance between the radiation source and the curable coating, the speed which the radiation source is passing a certain point in the coating, and the shape of the reflector for focusing the radiation, it is possible to optimize the properties of the cured coating. Properties possible to control are for example scratch resistance, black heel mark resistance, wear resistance and chemical resistance.

The electromagnetic radiation is applied after the coating has been allowed to physically dry during a time sufficient to obtain a physically dry coating as defined herein.

The cured coating is obtained by chemically curing the physically dry coating, by electromagnetic radiation. The wavelength of the electromagnetic radiation is matched with the initiator used and is commonly about 200-500 nm, although also other wavelengths are contemplated. The floor may be irradiated in any suitable way by an electromagnetic radiation source. In some embodiments the radiation source is mobile. The electromagnetic radiation source may be hand hold or mounted on a device. Such devices have for example been disclosed in WO0121321, U.S. Pat. No. 6,468,350, and EP1048362. Specific examples of suitable devices are marketed by Minuteman International Inc, UV-cured Inc and Balteschwiler AS. The radiation source may be continuous or pulsed and based on mercury tubes optionally doped with suitable metals, for example iron or gallium, electrode less, light emitting diodes, xenon technique, electric arc or electron beam or any other suitable electromagnetic radiation source that may initiate polymerisation.

One embodiment of the present invention relates to a method or technique for protecting and/or coating a floor, wherein a coating based on one or more radiation curable aqueous dispersion(s), comprising one or more polymerisable functional group(s) is applied to the floor, said coating forming a physically dry coating on said floor; and the physically dry coating is subjected to electromagnetic radiation whereby a cured coating is obtained. In some embodiments the coating is based on only one radiation curable aqueous dispersion. In some embodiments the coating is a wet coating. According to the present invention said physically dry coating has a pendulum hardness of about 10-120 König seconds (Ks) obtained according to standard DIN EN ISO 1522. Said physically dry coating is obtained by drying, such as at least partly evaporate water and volatile additives such as coalescents.

In one embodiment of the present invention the dispersion is a radiation curable aqueous polyurethane dispersion. In one embodiment of the present invention the dispersion is a radiation curable aqueous acrylate dispersion. In one embodiment of the present invention the dispersion is a mixture or a hybrid between a radiation curable aqueous acrylate and polyurethane dispersion. Said hybrid is a dispersion possessing both polyurethane and acrylate components, prepared in one batch. Said hybrid should not be mixed up with a physical mixture of the components.

In some embodiments of the present invention it is beneficial that not all water has evaporated before radiating the physically dry coating. In some embodiments of the present invention water is present in an amount sufficient to facilitate the mechanism of radiation curing.

In one embodiment a step of preparation of the floor is applied before applying the radiation curable aqueous dispersion to the floor.

In one embodiment a step of preparation of the floor is applied before applying the coating based on the radiation curable aqueous dispersion to the floor.

In one embodiment more than one layer of the coating based on a radiation curable aqueous dispersion, preferably a polyurethane or acrylate dispersion, is applied to a floor. It is preferred to apply 1, 2, 3, 4 or 5 layers to obtain the final coating. Each layer may be of a thickness of approximately 10-350 g wet/m². Thicknesses between 20-60 μm may also be used.

In one embodiment the floor may contain one or more coatings before applying the method according to the present invention. In one embodiment the floor contains no coating before applying the method according to the present invention.

The radiation curable aqueous polyurethane or acrylate dispersions according to the present invention are not limited to particular dispersions as long as they comprise one or more radiation polymerisable group(s) and result in a physically dry coating. Non-limiting examples of radiation polymerisable groups are acrylates, methacrylates, methacrylamides, and other vinyl derivatives, and thiolene components.

It must be possible to perform certain actions on the physically dry coating such as touching, walking or moving, such as rolling an electromagnetic radiation device on the floor without damaging the appearance and/or properties of said coating. In some embodiments the coating based on the aqueous dispersion is a radiation curable aqueous dispersion. Suitable radiation curable aqueous dispersions are radiation curable aqueous polyurethane dispersions and radiation curable aqueous acrylate dispersions, hybrids or mixtures thereof. In some embodiments a radiation curable aqueous polyurethane dispersion is preferred. Although the invention is not limited to certain coatings based on radiation curable aqueous polyurethane dispersions non-limiting examples thereof are Bayhydrol UV VPLS 2280, Setaqua™ X11751 and Bona UVPUD47.

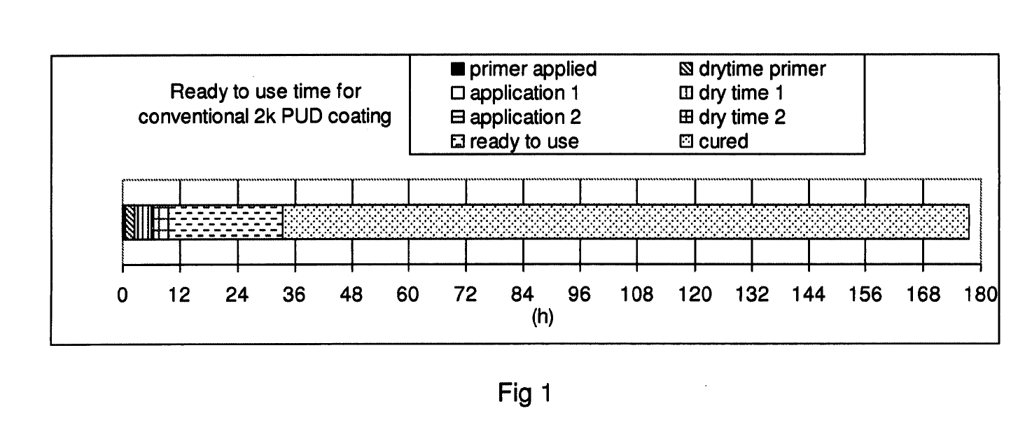

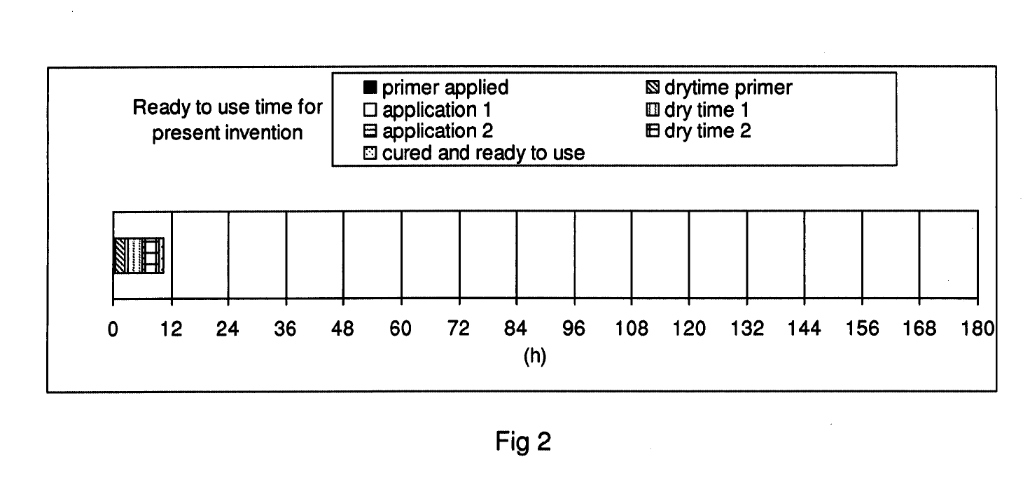

In the experimental section an example prepared according to a method of the present invention is compared with a traditional method. Both coatings have similar final properties but the one obtained according to the present invention is obtained much quicker and may be beneficial, for the skilled person applying the coating to the floor to use. The quality of the coating obtained and the time consumed for restoring a floor according to the present invention has been compared with a traditional method, where a two component polyurethane dispersion with an associated isocyanate hardener (2k PUD) (traditionally polyurethane dispersions are marketed together with an associated reactive hardener) has been used. FIG. 1 illustrates an average time to obtain a cured coating with a 2k PUD under standard conditions. FIG. 2 illustrates that the present innovation can give a high quality coating in much shorter time under similar conditions as used to obtain the 2k PUD. The floor coated with 2k PUD may be used after 34 hours and 20 minutes, but the desired properties, relating to scratch resistance, black heel mark resistance, wear resistance, water resistance and chemical resistance, of the coating takes about 178 hours to develop.

By using the method according to the present invention it is possible to obtain a cured and functional coating that has obtained desired properties, such as scratch resistance, black heel mark resistance, wear resistance and chemical resistance after only about 11 hours. Additionally, the coating will post develop and properties improve over the first day. Most importantly, the properties developed after applying the electromagnetic radiation, are more than sufficient to start using the floor.

### EXAMPLES

The following examples are illustrative examples only and should not in any way be interpreted as limiting to the invention.

### Example 1

### Comparative

Traditional coating of a floor, of 50 m², was performed using a two component polyurethane finish, Aquapur HPX, (2k PUD) as a topcoat.

In this example the flooring material, wood, was prepared with an adequate technique to obtain a surface free from old coatings, dust and dirt. An aqueous primer was applied to the clean surface to achieve good adhesion to the flooring material. This action took about 25 minutes to complete. The primer had a drying time of 2 hours. Thereafter the flooring surface was sanded for 35 minutes. The sanded areas were then vacuum cleaned and wiped off with a moist cloth. This action took about 30 minutes to complete. Before applying the two layers of top coat the 2k finish had to be prepared by mixing the PUD with the reactive hardener (which is determined by the top coat used, normally each top coat comes with a suitable reactive hardener) and the first layer of coating was applied with a roller, which took about 25 minutes to be completed. After 3 hours the surface was physically dry and the second layer of the same coating was applied, using the same equipment as for the first layer (about 25 minutes to be completed). After further 3 hours, counting 10 hours 20 minutes from start, the coating was physically dry but had insufficient properties, such as pendulum hardness, scratch resistance, black heel mark resistance, wear resistance, water resistance and chemical resistance. The coating continued to dry and cure for another 24 hours (a total of 34 h and 20 min) and the floor owner can not until after this time start to use the floor e.g. moving in furniture, but not carpets, and traffic the surface but the coating had not developed its final properties. After an additional 144 hour the coating was considered to have reached its desired properties and the surface may be covered with carpets and is possible to clean with a wet cloth. The coating, had after this time, obtained desired properties regarding pendulum hardness, scratch resistance, black heel mark resistance, wear resistance, water resistance and chemical resistance. The time from start to when a limited usage of the floor is possible is 34 hours and 20 minutes. To obtain a coating ready for heavy usage the total time is 178 hours and 20 minutes.

### Example 2

### An Embodiment of the Present Invention

A floor of 50 m² is coated by a method of the present invention.

In the example the flooring material, wood, was prepared with an adequate technique to obtain a surface free from old coatings, dust and dirt. The mobile UV-curing device used hade a medium pressure mercury lamp with an arc length of 38 cm and an output effect of 80 W/cm. The device was transported in a speed of 8 m/minute during the curing process. A primer was applied to the surface to achieve good adhesion to the flooring material which took about 25 minutes to complete. The primer had a drying time of 2 hours. Thereafter, the surface area was sanded for 35 minutes. The sanded areas were then vacuum cleaned and wiped of with a moist cloth. This action took about 30 minutes to complete. A finish based on an aqueous polyurethane dispersion with polymerisable acrylate and methacrylate groups, was applied with a roller which took about 30 minutes to complete. After about 3 hours the surface was physically dry and the second layer of the aqueous polyurethane dispersions with polymerisable groups was applied using the same equipment as for the first layer. This step took about 30 minutes to complete. After 3 hours, the coating was physically dry but had not fully developed its desired properties regarding pendulum hardness, scratch resistance, black heel mark resistance, wear resistance, water resistance and chemical resistance, yet the coating was sufficiently dry to allow walking, and transportation of the mobile electromagnetic radiation device without damaging its appearance and/or properties. The coating was cured with the mobile electromagnetic radiation device which was transported over the entire surface. The curing operation took about 35 minutes and immediately after the radiation device had irradiated the area, a coating having the desired properites, such as high pendulum hardness, high scratch resistance, high black heel mark resistance, high wear resistance, high water resistance and high chemical resistance was obtained. The room with the newly coated floor may be furnished after completion of the radiation operation. The floor may be covered with carpets, trafficed, and, if desired, cleaned with a wet cloth. The time from start to a point when the floor coating has desired properties and it is possible to use the floor without restrictions is about 11 hours and 5 minutes.

TABLE 1
König pendulum hardness of coatings, for
2k PUD and Bona UVPUD47 after different times and
directly after radiation curing of Bona UVPUD47.

| | 2k PUD | Bona UVPUD47 |
|---|---|---|
| | (Ks) | (Ks) |
| König pendulum hardness after 3 hours | 30 | 24 |
| König pendulum hardness after 3 hours and | N/A | 115 |
| after radiation curing | | |
| König pendulum hardness after 155 hours | 80 | 150 |

### Example 3

### An Embodiment of the Present Invention

In the following test the method of the invention was compared with a prior art system using a waterborne two component coating, which is a waterborne coating system with very good properties such as scratch resistance, black heel mark resistance, wear resistance, water resistance and chemical resistance, but time consuming. The scratch resistance test was made on a coating applied to a glass plate in a thickness of 120 nm. After drying and curing, the surface was scratched with a fingernail and visually inspected. When the coating was easy to scratch it was rated as 1 on a scale from 1 to 5, and when it was not possible to scratch the coating it was rated as 5. The black heel mark test was made on a coating applied to a glass plate in a thickness of 120 nm. After drying, and curing, the surface was hit with a piece of rubber at low angle. Rating of the black heel marks resistance is dependent on the possibility of removing the mark, or how easy it is to remove the residue of rubber from the surface using the same piece of rubber as an eraser. A rubber mark not possible to remove was rated as 1 on a scale from 1 to 5, and an easily removed rubber mark was rated as a five.

Wear resistance was performed according to European standard method ENV 13696 with the grid feeder system.

The water resistance test was made on a coating applied to a glass plate in a thickness of 120 nm. After additional time, and curing a droplet of water was applied to the surface and an evaporation protection was mounted. After 120 minutes the water was wiped off and the coating inspected to determine possible haziness and deformations. A slightly hazed or deformed coating was rated as 1 on a scale from 1 to 5, and an unaffected was rated as 5.

Chemical resistance test was performed by applying a coating having a thickness of 120 nm to a glass plate. After drying and curing, a drop of ethanol (99%), acetic acid (20% in water) and alkaline polish remover “Fernox” (10% in water), respectively, was applied to the surface. The drops were covered with a small cup to prevent evaporation. After 120 minutes, the chemicals was collected and the coating inspected for marks and/or deformations. A slightly marked or deformed coating was rated as a 1 on a scale from 1 to 5, and an unaffected was rated as a 5.

TABLE 2
Properties of the coatings, 2k PUD and Bona UVPUD47,
3 hours after applying the coatings and directly after
radiation curing of Bona UVPUD47. The properties are
tested according to methods described above.

| | | Bona |
|---|---|---|
| | 2k PUD | UVPUD47 |
| Scratch resistance | 1 | 5 |
| Black heel marks resistance | 1 | 5 |
| Wear resistance (mg/100 r) | not possible to | 3.5 |
| | test | |
| Water resistance | 1 | 5 |
| Chemical resistance | 1 | 5 |

5 = excellent 1 = poor

TABLE 3
Properties of the coatings, 2k PUD and Bona UVPUD47, 155 hours
after applying the coatings. Bona UVPUD47 was radiation cured
3 hours after being applied to the substrate. The properties
are tested according to methods described above.

| | | Bona |
|---|---|---|
| | 2k PUD | UVPUD47 |
| Scratch resistance | 3 | 5 |
| Black heel marks resistance | 4 | 5 |
| Wear resistance (mg/100 r) | 2.8 | 3.5 |
| Water resistance | 4 | 5 |
| Chemical resistance | 4 | 5 |

5 = excellent 1 = poor

### Example 4

### An Embodiment of the Present Invention Using a Finish

In this example, the substrate is a worn floor, of 12 m², initially coated industrially with a 100% UV curing coating. To this substrate, it is very difficult to get good adhesion. The floor was cleaned. The floor was coated and cured essentially as in example 2 except for the addition of reactive additive based on polyisocyanates. To 1.425 l base finish Bona 424-516 G was added 0.075 l Bona Hardener Traffic/Flow and the mixture was shaken for a few minutes to assure homogeneity.

The time to clean, coat, and cure the finish was marginally different from specifications in example 2. The properties of the coating are equal or better than the system of example 2 with the exception of adhesion to difficult substrates which is much improved by the coating obtained according to example 4.

### Example 5

### The Adhesion of a Coating to a Difficult Substrate is Evaluated with and without Reactive Additive

To 1 kg of the UV curing dispersion Bona UVPUD47, 50 g of a 65% solution of the polyisocyanate Easaqua XM 502 in DMM (dipropylene glycol dimethyl ether) (18.3% isocyanate)) was added and the mixture shaken for about 30 s. The finish is thereafter applied to an industrially coated and UV cured wood tile from Boen. Water is allowed to evaporate and the surface is exposed to UV light, according to the method disclosed in example 2, and the cured coating is examined. The coating properties are reported in Table 4:

The coin test: the surface is scratched with a hand hold coin and the force needed to detach fragments of the coating is used as an indication of the adhesion.

The knife test: a V-shaped mark is cut in the film and the film is pealed of by the tip. The ease of detachment is taken as a measure of adhesion.

TABLE 4

| | Right after UV cure | | 16 h after UV cure | |
|---|---|---|---|---|
| | | With | | With |
| | No promotor | promotor | No promotor | promotor |
| Coin test | 1 | 4 | 1 | 5 |
| Knife test | 1 | 4 | 1 | 5 |

Legend: 1 = inadecquat performance, 5 = excellent performance.

Coating properties such as black heal mark resistance, scratch resistance, and chemical resistance is high and essentially unaffected by the addition of adhesion promoter.

### Example 6

### Role of Additive Promoting Adhesion

Varying amounts of the adhesion promoter (BONA 383-15M) was added to the UV curing dispersion (BONA 155-74-5) according to the table below. The mixtures were used to coat a substrate and evaluated as above after UV-curing.

TABLE 5

| %-age adhesion | | |
|---|---|---|
| promotor | Coin test | Knife test |
| 0% | Poor adhesion. | White lines along cut |
| | | marks and easy to peel off |
| | | film. |
| 1% | Poor adhesion | White lines but narrower. |
| 2% | Inprooved adhesion | No white lines along cut |
| | but the film can be | marks but the film can be |
| | scrached off. | peeled off. |
| 3% | Excellent adhesion, | Good adhesion, the film |
| | the film can not be | can not be scrached off 1 |
| | scrached off. | day after application. |
| 6% | As for 3%. | Excellent adhesion, the |
| | | film can not be scrached of |
| | | even directly after cure. |
| | | After 1 day, 3 and 6% |
| | | gives the same result. |
| 9% | As for 3%. | As above. Slightly better |
| | | adhesion directly after |
| | | cure. No difference |
| | | between 3, 6, and 9% one |
| | | day after cure. |

### Example 7, an Embodiment of the Present Invention Using a Finish with a Reactive Additive with Acrylate Functionality

To the finish Bona 424-16 C was added 1.5% by weight of Bona acrylated adhesion promotor, prepared according to example 9. The finish was applied to an industrially coated wood tile from Boen and was cured by UV light, 2 h after evaporation of water. The coating showed properties (scratch, alcohol, water, and Fernox (basic cleaner) resistance, black heel mark resistance, adhesion using the coin test identical to the finish obtained in Example 5, but adhesion according to the knife test was improved.

### Example 8, an Embodiment of the Present Invention Using a Finish with a Curing Additive with Acrylate Functionality

Example 8 was performed in line with Example 6 but the acrylated adhesion promotor was prepared according to example 9. The results were similar to the results obtained in Example 6.

### Example 9, Synthesis of Acrylated Adhesion Promotor

To Bona Naturale Hardener (400 g) was added 2-hydroxyethyl acrylate (28.9 g) and the catalyst metatin (2 drops). The mixture was stirred and heated to 80° C. for 2 h. The isocyanate number was then 7,20% and the acrylate density 0.58 mmol/g. The product was cooled and used as such.

### Example 10, Synthesis of Acrylated Adhesion Promotor

To Bona Naturale Hardener (375 g) was added 2-hydroxyethyl acrylate (37,9 g) and the catalyst metatin (2 drops). The mixture was stirred and heated to 80° C. for 2 h. The isocyanate number was then 6,10% and the acrylate density was 0.81 mmol/g. The product was cooled and used as such.

1. A method for coating a floor, wherein said method comprises applying a coating based on one or more radiation curable aqueous dispersion(s) comprising one or more polymerisable functional group(s) to a floor;
drying said coating to obtain a physically dry coating; and
subjecting the physically dry coating to electromagnetic radiation,
whereby a cured coating is obtained.

2. The method according to claim 1, wherein the radiation curable aqueous dispersion is selected from the group consisting of a radiation curable polyurethane dispersion, a radiation curable acrylate dispersion, and a hybride or a mixture thereof.

3. The method according to claim 1, wherein the electromagnetic radiation is UV radiation having a wavelength between about 200-500 nm.

4. The method according to claim 1, wherein the floor is cleaned and/or undressed before applying said coating.

5. The method according to claim 1 wherein said one or more radiation curable aqueous dispersion(s) comprises one or more additive.

6. The method according to claim 1, wherein a reactive additive is added.

7. The method according to claim 6, wherein said reactive additive is selected from the group consisting of a poly- or oligoisocyanate based additive, an aziridine based additive, a silicon derivative, a silicon product having reactive functionalities, old a carbodiimide additive, and any combination thereof.

8. The method of according to claim 7, wherein the poly- or oligoisocyanate based additive comprises one or more additional functional group(s).

9. The method of claim 7, wherein said silicon additive is a radically polymerisable silicon derivative and said silicon product having reactive functionalities is selected from the group consisting of a silica sol, silica particles treated with an unsaturated alkoxysilane, and a mercaptosilane.

10. The method of claim 9, wherein said radically polymerisable silicon derivative is selected from 3-(trimethoxysilyl)propyl methacrylate, 3-(triethoxysilyl)propyl methacrylate, vinyltrimethoxysilane, vinyltriethoxysilane, allyltrimethoxysilane, (methacryloxymethyl) methyldimethoxysilane, and an adduct of an aminosilane and an acrylate; and said silica sol or silica particles treated with an unsaturated alkoxysilane.

11. The method of claim 10, wherein said aminosilane is one of 3-(trimethoxysilyl)propylamine, 3-(triethoxysilyl)propylamine, and (dimethoxymethylsilyl) methylamine; and said acrylate is one of tripropylene glycol diacrylate, trimethylolpropane triacrylate, and pentaerythritol pentaethoxylate tetraacrylate.

12. The method according to claim 1, wherein the floor is selected from one of biologically derived flooring materials and mineral derived flooring materials.

13. The method according to claim 12, wherein the biologically derived flooring material is selected from at least one of woods, cork, and bamboos; and the mineral derived flooring material is selected from one of concrete, terrazzo, ceramic tile, grout, and plastics.

14. The method according to claim 1, wherein one or more polymerisable functional group(s) is selected from acrylates, methacrylates, methacrylamides, other vinyl derivatives, and thiolene components.

15. A reactive additive wherein the reactive additive is one of a poly- or oligoisocyanate based additive having one or more additional reactive group(s).

16. The reactive hardener according to claim 15, wherein said one or more additional reactive group(s) is selected from an acrylate group and a silyl group.

17. A coated floor obtainable by a method according to claim 1.

18. The floor according to claim 17, wherein the floor is at least one of cleaned and undressed before applying said coating.
